(19) United States
(12) Patent Application Publication
(10) Pub. No.: US 2007/0255368 A1
(43) Pub. Date: Nov. 1, 2007

(54) Implantable medical electrical stimulation lead with distal fixation and method

(76) Inventors: Eric H. Bonde, Minnetonka, MN (US); Eric M. Stetz, Lino Lakes, MN (US); Carole A. Tronnes, Stillwater, MN (US); James T. Henry, Blaine, MN (US)

Correspondence Address: DICKE, BILLIG & CZAJA, FIFTH STREET TOWERS, 100 SOUTH FIFTH STREET, SUITE 2250, MINNEAPOLIS, MN, 55402, US

(21) Appl. No.: 11/413,354

(22) Filed: Apr. 28, 2006

(51) Int. Cl. A61N 1/00

(52) U.S. Cl. 607/116

(57) ABSTRACT

An implantable medical electrical lead for applying electrical stimulation energy from an external power source and adapted to be introduced through a small diameter needle. The lead includes a lead body and a fixation assembly. The lead body includes a wire and an electrically non-conductive material. The wire forms a wound coil along a distal portion thereof. The non-conductive material covers some of the wire coil, with an uncovered distal region of the wire serving as an electrode. The fixation assembly is coupled to the uncovered distal region and includes at least one fixation member. The fixation assembly is transitionable between a contracted state and an expanded state, with the fixation member extending outwardly relative to the coil to a greater extent in the expanded state. In the expanded state, the fixation assembly serves to inhibit axial migration of the lead body.

### BACKGROUND OF THE INVENTION

The present invention relates to systems and methods for providing electrical stimulation to bodily tissue, such as electrically stimulating a portion of a patient's nervous system. More particularly, it relates to temporarily implantable electrical stimulation leads, such as a peripheral nerve evaluation lead used to stimulate a sacral nerve, with enhanced resistance to migration, and related systems and methods of use.

A number of human bodily functions are affected by the nervous system. For example, bodily disorders, such as urinary incontinence, urinary urge/frequency, urinary retention, pelvic pain, bowel dysfunction (constipation, diarrhea, etc.), erectile dysfunction, etc., are all bodily functions influenced by the sacral nerves. As a point of reference, urinary incontinence is the involuntary loss of control over the bladder. Incontinence is primarily treated through pharmaceuticals and surgery. Many pharmaceuticals do not adequately resolve the issue and can cause unwanted side effects; further, a number of surgical procedures have a low success rate and/or are not reversible. Similar treatment insufficiencies have likewise been noted for many of the other maladies previously mentioned.

As an alternative to conventional pharmaceuticals and/or invasive surgical procedures, neurostimulation has more recently been recognized as a viable treatment approach for many patients. By way of background, the organs involved in bladder, bowel, and sexual function receive much of their control via the second, third, and fourth sacral nerves, commonly referred to as S2, S3, and S4, respectively. Electrical stimulation of these various nerves has been found to offer some control over these functions. Several electrical stimulation techniques have been suggested, including stimulation of nerve bundles within the sacrum. Regardless, in order to consistently deliver electrical stimulation to the sacral nerve(s), certain anatomical obstacles must be addressed. The sacrum is a large, triangular bone situated at the lower part of the vertebral column, and at the upper and back part of the pelvic cavity. The spinal canal runs through the greater part of the sacrum. Further, the sacrum is perforated by the anterior and posterior sacral foramina though which the sacral nerves pass.

With the above anatomical description in mind, partial control over one or more of the functions (or dysfunctions) previously mentioned has been achieved by implanting a neurostimulation lead at or near the sacral nerves. As a point of reference, other nerve(s) or tissue can similarly be electrically stimulated to produce different effects. Relative to sacral nerve stimulation, however, the neurostimulation lead, having several stimulation electrodes, can be permanently implanted within and/or anteriorly beyond the sacral foramen at which the sacral nerve in question is anatomically located. Because the lead, and in particular the stimulation electrodes, must remain in operative proximity to the sacral nerve, the permanent lead (sometimes referred to as a “chronic lead”) can be sutured within the patient's body to resist migration. In light of the invasive nature associated with this approach, minimally invasive neurostimulation leads have been developed, incorporating features proximal the electrodes that inhibit migration and/or retrograde dislodgement. Permanent leads of this type are typically somewhat sizable to not only present a sufficient number of electrodes, but to also better resist migration. Regardless, wire cabling extending from the lead is placed in a subcutaneous tunnel, and connected to a subcutaneously-implanted pulse generator. One example of such a system is available from Medtronic, Inc., of Minneapolis, Minn. under the trade name InterStim®. Other chronic leads/systems are further described in U.S. Pat. Nos. 6,999,819; 6,971,393; and 6,847,849, each commonly assigned to the assignee of the present invention and the teachings of all of which are incorporated herein by reference.

Some patients may view the permanent neurostimulation lead and related pulse generator implantation described above as being a fairly traumatic procedure. Thus, efforts are conventionally made to ascertain in advance whether the patient in question is likely to receive benefit from sacral nerve stimulation. In general terms, the test stimulation procedure entails the temporary implantation of a neurostimulation lead in conjunction with an externally carried pulse generator or other power source. Once in place, the patient is exposed to neurostimulation over a trial period (e.g., 3-7 days) during which the patient can experience the sensation of nerve stimulation during various everyday activities, as well as recording the changes, if any, in the bodily dysfunction of concern (e.g., a patient experiencing urinary incontinence can maintain a voiding diary to record voiding behavior and symptoms with the stimulation). The record of events is then compared with a base line and post-test stimulation diaries to determine the effect, if any, of sacral nerve stimulation on the symptoms being experienced by the patient. If the test stimulation is successful, the patient and his/her clinician can make a better informed decision as to whether permanent implantation and long-term sacral nerve stimulation is a viable therapy option.

Temporary implantation of the neurostimulation lead is normally done in one of two manners. With one approach, sometimes referred to as a “staged implantation,” a conventional, permanent or chronic neurostimulation lead is implanted at the desired sacral location, with the cable carrying the coiled conductor wiring being externally extended through the patient's skin and coupled to the pulse generator. While viable, this technique entails the use of surgical equipment normally employed to permanently implant the stimulation lead. By way of background, implantation of a permanent sacral nerve stimulation lead normally requires the use of a fairly large introducer (e.g., an elongated, 13 gauge tube), and the chronic stimulation lead has a fairly large diameter. While local and/or general anesthesia is available, some patients may be apprehensive to participate in a short-term test of this type in view of the size of the instrument(s)/stimulation lead.

To better address the reluctance of some patients to participate in the stimulation test procedure described above, a second technique has been developed that entails the use of a smaller diameter, more simplified neurostimulation lead intended to be implanted on only a temporary basis. In general terms, the temporary stimulation lead (sometimes referred to as a peripheral nerve evaluation lead or “PNE” lead) has a single electrode and is of sufficiently small diameter so as to be percutaneously inserted using a small diameter needle (e.g., a 20 gauge needle). Many patients are not overly threatened by a small diameter needle and thus are more likely to participate in the trial stimulation. The percutaneous test stimulation is similar to an epidural nerve block, except that the temporary lead is inserted and left in the patient's back during the trial. The end of the lead that remains on the outside of the patient's body is secured to the patient's skin with, for example, surgical tape. Upon conclusion of the trial stimulation, the lead is removed from the patient.

While generally preferred by patients, the percutaneous, PNE lead technique may have certain drawbacks. For example, while the temporary simulation lead is highly capable of delivering the necessary stimulation energy throughout the evaluation period, it is possible that the lead may migrate. For example, any pulling or tugging on the proximal end of the lead body (from outside of the patient's body) could be directly communicated to the lead's electrode, thus creating a higher likelihood of electrode dislodgement and poor stimulation. Efforts have been made to address this concern, for example as described in U.S. Pat. No. 6,104,960, the teachings of which are incorporated herein by reference and assigned to the assignee of the present invention. In particular, a temporary neurostimulation lead is described as having a coiled configuration that better accommodates axial forces placed onto the lead body (e.g., tugging or pulling on the proximal end of the lead body). Any additional efforts to further minimize migration of the temporary neurostimulation lead would be well received, not only in the one exemplary context of peripheral sacral nerve electrical stimulation, but also for any other procedure in which an implantable medical electrical stimulation lead is used.

In light of the above, a need exists for a medical electrical lead which may be safely and effectively implanted in a minimally invasive manner, but which better inhibits axial migration of dislodgement of the lead body from the stimulation site, such as a sacral location.

### SUMMARY OF THE INVENTION

Some aspects in accordance with principles of the present invention relate to an implantable medical electrical lead for applying electrical stimulation energy to bodily tissue of a patient from a power source located external the patient, the lead adapted to be introduced through, and released from, a needle having a lumen defining a diameter of no greater than 0.05 inch. With this in mind, the lead includes a lead body and a fixation assembly. The lead body includes a wire and an electrically non-conductive material. The wire defines a distal portion terminating at a distal end and a proximal portion terminating at a proximal end. The wire forms a wound coil along at least the distal portion. Further, the proximal end of the wire is adapted to be electrically coupled to a power source. The non-conductive material covers at least a section of the distal portion, terminating proximal the distal end of the wire coil. With this arrangement, an uncovered distal region of the wire coil is defined, characterized by the absence of the non-conductive material, with at least a segment of the uncovered distal region serving as a lead electrode. The fixation assembly is coupled to the uncovered distal region and includes at least one fixation member. In this regard, the fixation assembly is configured and assembled to the wire coil so as to define, and be transitionable between, a first, contracted state and a second, expanded state. An amount or level of radial extension of the fixation member differs between the two states, with the fixation member extending radially outwardly relative to the wire coil to a greater extent in the expanded state as compared to the contracted state. In the expanded state, then, the fixation assembly serves to inhibit axial dislodgement of the lead body following implant, especially in an area of the electrode. In some embodiments, the fixation member is formed by a suture, pliable polymeric material, or a sponge material; and in related embodiments, a plurality of the so-formed fixation members are provided. In other embodiments, the fixation assembly includes a cap mounted to the distal end of the wire coil and configured to capture the fixation member relative to the wire coil.

Other aspects in accordance with principles of the present invention relate to a system for providing medical electrical stimulation to bodily tissue of a patient from a power source located external the patient. The system includes a hollow needle and an implantable medical electrical lead. The needle defines a lumen having a diameter of not more than 0.05 inch, and in some embodiments forms a sharpened needle tip. The lead is slidably disposed within the needle lumen and includes a lead body and a fixation assembly. The lead body includes a wire and an electrically non-conductive material. The wire defines a distal portion terminating at a distal end and a proximal portion terminating at a proximal end. The wire forms a wound coil along at least the distal portion. Further, the proximal end of the wire is adapted to be electrically coupled to a power source. The non-conductive material covers at least a section of the distal portion, terminating proximal the distal end of the wire coil. With this arrangement, an uncovered distal region of the wire coil is defined, characterized by the absence of the non-conductive material, with at least a segment of the uncovered distal region serving as a lead electrode. The fixation assembly is coupled to the uncovered distal region and includes at least one fixation member. In this regard, the fixation assembly is configured and assembled to the wire coil so as to define, and be transitionable between, a first, contracted state and a second, expanded state. An amount or level of radial extension of the fixation member differs between the two states, with the fixation member extending radially outwardly relative to the wire coil to a greater extent in the expanded state as compared to the contracted state. In some embodiments, the needle is a 20 gauge needle. In other embodiments, the fixation assembly is configured to be forced to the contracted state when the lead body is inserted within the needle lumen. In other embodiments, the system is configured for performing a sacral peripheral nerve stimulation procedure such that the lead body is a PNE lead and the needle is adapted to percutaneously access a sacral foramen.

Yet other aspects in accordance with principles of the present invention relate to a method of providing electrical stimulation to bodily tissue of a patient at a stimulation site via a power source external the patient. The method includes providing an implantable medical electrical lead including a lead body and a fixation assembly. The lead body includes a wire and an electrically non-conductive material. The wire defines a distal portion terminating at a distal end and a proximal portion terminating at a proximal end. The wire forms a wound coil along at least the distal portion. The non-conductive material covers at least a section of the distal portion, terminating proximal the distal end of the wire coil. With this arrangement, an uncovered distal region of the wire coil is defined, characterized by the absence of the non-conductive material, with at least a segment of the uncovered distal region serving as a lead electrode. The fixation assembly is coupled to the uncovered distal region and includes at least one fixation member. In this regard, the fixation assembly is configured and assembled to the wire coil so as to define, and be transitionable between, a first, contracted state and a second, expanded state, with a radially outward extension of the fixation member relative to the wire coil being greater in the expanded state. The lead body is slidably disposed within a needle lumen having a diameter of no greater than 0.05 inch. In this regard, the fixation assembly is in the contracted state when the lead body is within the needle lumen. A distal tip of the needle is percutaneously directed toward the stimulation site. The lead body is deployed from the distal tip to implant the lead body at the stimulation site. The fixation assembly transitions from the contracted state to the expanded state. The needle is proximally withdrawn from the lead such that the proximal portion of the wire is external the patient. The proximal end of the wire is electrically coupled to a power source external the patient. In this regard, following implantation, the fixation assembly in the expanded state inhibits axial retrograde migration of the lead body from the stimulation site. In some embodiments, the fixation assembly self-transitions to the expanded state by the fixation member absorbing bodily fluids. In other embodiments, the fixation assembly self-transitions to the expanded state by the fixation member being released relative to the wire coil once the lead body exits the needle lumen. In yet other embodiments, the method is performed as part of a peripheral sacral nerve stimulation procedure, with the distal tip of the needle being directed into a sacral foramen.

### BRIEF DESCRIPTION OF THE DRAWINGS

FIG. 1 is a simplified plan view of a system for providing electrical stimulation to bodily tissue of a patient, including a medical electrical lead in accordance with principles of the present invention;

FIG. 2 is an enlarged side view of a portion of one embodiment of the lead of FIG. 1;

FIG. 3 is a cross-sectional view of the lead portion of FIG. 2, taken along the lines 3-3;

FIG. 4A is a side view of the lead of FIG. 2, including a fixation assembly in a contracted state;

FIG. 4B is a side view of the lead of FIG. 2 with the fixation assembly in an expanded state;

FIG. 5A is a simplified cross-sectional view of a portion of an alternative embodiment medical electrical lead in accordance with principles of the present invention;

FIG. 5B is a simplified cross-sectional view of a portion of another alternative embodiment medical electrical lead in accordance with principles of the present invention;

FIG. 5C is a simplified cross-sectional view of a portion of another alternative embodiment medical electrical lead in accordance with principles of the present invention;

FIG. 5D is a simplified cross-sectional view of a portion of another alternative embodiment medical electrical lead in accordance with principles of the present invention;

FIG. 6A is a posterior view of a human patient's spinal column showing a location of a sacrum relative to an outline of a body of the patient;

FIG. 6B is a simplified sectional view of a human anatomy in a region of the sacrum;

FIG. 7 is a flow diagram of a method of providing stimulation energy to bodily tissue of a patient in accordance with principles of the present invention; and

FIGS. 8A-8C illustrate several steps associated with the method of FIG. 7.

### DETAILED DESCRIPTION OF THE INVENTION

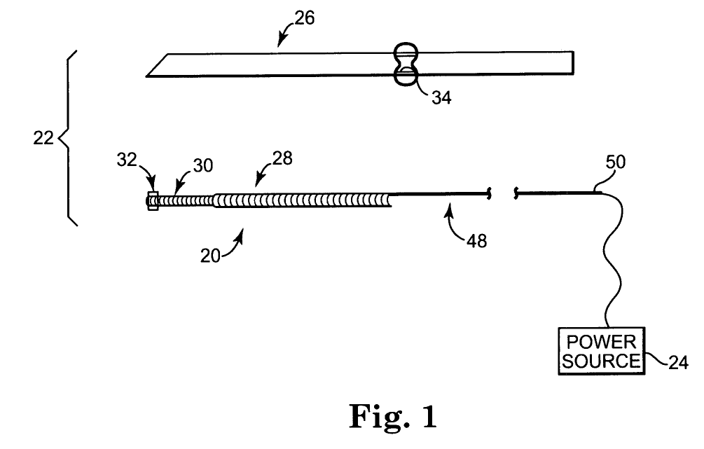

One embodiment of an implantable medical electrical lead 20 in accordance with principles of the present invention is shown in simplified form in FIG. 1 as part of a system 22 for delivering stimulation energy to bodily tissue of a patient (not shown) via a power source 24 (e.g., a pulse generator) maintained external the patient. The system 22 can incorporate components in addition to those illustrated, and includes the lead 20 and a needle 26. The lead 20 includes a flexible lead body 28, otherwise forming an electrode 30, and a fixation assembly 32 (shown schematically). Details on the various components are provided below. In general terms, however, the lead 20 is sized to be slidably received within a small diameter lumen 34 (referenced generally) of the needle 26 for percutaneous delivery to a stimulation site. The fixation assembly 32 defines a contracted state when disposed within the needle lumen 34, and transitions to an expanded state following deployment from the needle 26. In the expanded state, the fixation assembly 32 inhibits migration of the lead 20, and in particular the lead body 28, from the implantation (or stimulation) site. Further, energy from the external power source 24 can be conducted to the electrode 30 to effectuate desired tissue stimulation, for example stimulation of a peripheral sacral nerve.

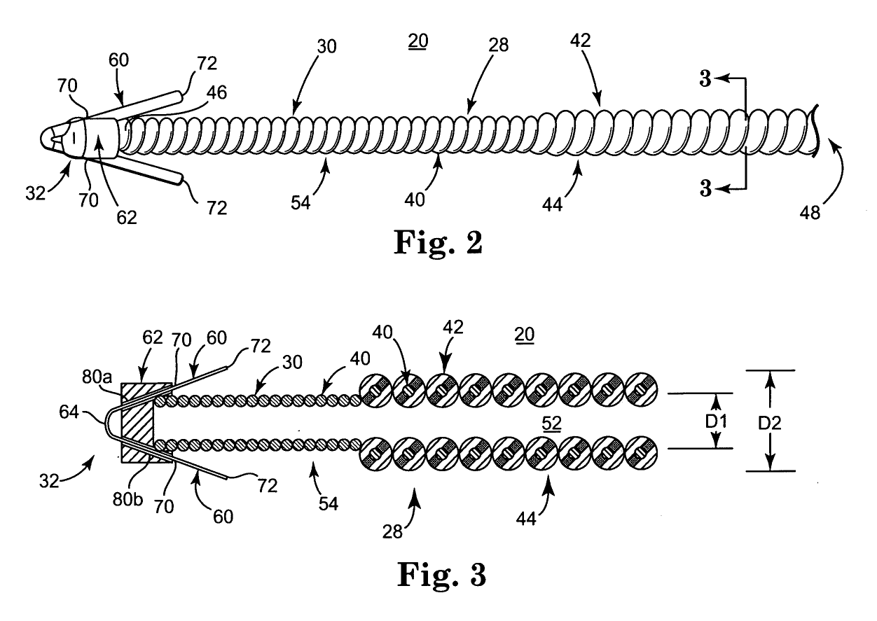

One embodiment of the lead 20 in accordance with principles of the present invention is shown in greater detail in FIG. 2. Once again, the lead 20 includes the lead body 28 and the fixation assembly 32. As described below, the fixation assembly 32 is coupled to the lead body 28 at or distal the electrode 30 to provide a more positive resistance to migration/movement of the lead body 28 in a region of the electrode 30 as compared to conventional designs that either do not provide a fixation assembly or position an anchor of some type proximal the electrodes.

The lead body 28 is, in one embodiment, akin to a PNE lead having a relatively small maximum outer diameter (e.g., not greater than 0.05 inch, more preferably not greater than 0.04 inch, even more preferably not greater than 0.03 inch, and in one embodiment on the order of 0.025 inch, although other dimensions are also acceptable), such that the lead 20 can be implanted using a small diameter needle (e.g., the needle lumen 34 (FIG. 1) can have a diameter corresponding with the outer diameters specified above, for example as found with conventional 20 gauge or 19 gauge foramen needles). With this in mind, the lead body 28 of FIG. 2 includes a wire 40 and an electrically non-conductive material 42 covering a section of the wire 40 as described below. The wire 40 is formed of an electrically conductive material (e.g., stainless steel such as SST 316L stainless steel multi filament wire, MP35N alloy, etc.), and defines a distal portion 44 terminating at a distal end 46, and a proximal portion 48 terminating at a proximal end 50 (best shown in FIG. 1). The wire 40 forms a wound coil along at least the distal portion 44 as shown in FIG. 2 and well as in FIG. 3. The wire coil 40 can be closely wound as shown, or in alternative embodiments, individual windings of the wire coil 40 can be longitudinally spaced. The coiled nature of the wire 40 can further be continued along a majority of the wire's length, for example including a distal segment of the proximal portion 48. Alternatively, the wire 40 can be relatively straight or non-coiled along portions proximal the distal portion 44, including, for example, the proximal end 50 that can otherwise form or be assembled to a connector pin or element (not shown) that facilitates electrical connection to the power source 24 (FIG. 1). Regardless, the coil configuration of the wire 40 along the distal portion 44 imparts a longitudinal strain relief attribute to the lead body 28 such that any tugging or pulling on the proximal portion 48 will not automatically be translated to the electrode 30. Further, as shown, the coil configuration generates or defines an internal passage 52 (FIG. 3) along at least the distal portion 44.

The electrically non-conductive material or insulator 42 is disposed, formed, or coated over sections of the wire 40, and can assume a variety of forms. For example, the non-conductive material 42 can be ETFE (a polymer of tetrafluoroethlyene and ethylene), PTFE, polyurethane, fluoropolymer, silicone rubber, polyester, etc. Regardless, the non-conductive material 42 preferably encompasses a majority of the wire 40, including the proximal portion 48 except at and adjacent the proximal end 50 (to allow electrical coupling of the proximal end to the power source 24 (FIG. 1)). Further, as shown in FIGS. 2 and 3, the non-conductive material 42 terminates proximal the distal end 46 of the wire 40, resulting in an uncovered distal region 54. At least a segment of this uncovered distal region 54 serves as the lead electrode 30 (e.g., areas of the uncovered distal region 54 that are not otherwise electrically insulated by the fixation assembly 32 as described below).

As best shown in FIG. 3, the non-conductive material 42 has a thickness (exaggerated in the view of FIG. 3), such that an overall diameter of the lead body 28 along the uncovered distal region 54 is slightly reduced. For example, in one embodiment, a diameter D1 at the uncovered distal region 54 is less than a diameter D2 defined by a combination of the wire 40 and the non-conductive material 42 by approximately 0.001-0.010 inch, preferably on the order of 0.002-0.006 inch. Regardless, in one embodiment this thickness differential provides a spacing for locating component(s) of the fixation assembly 32 as described below.

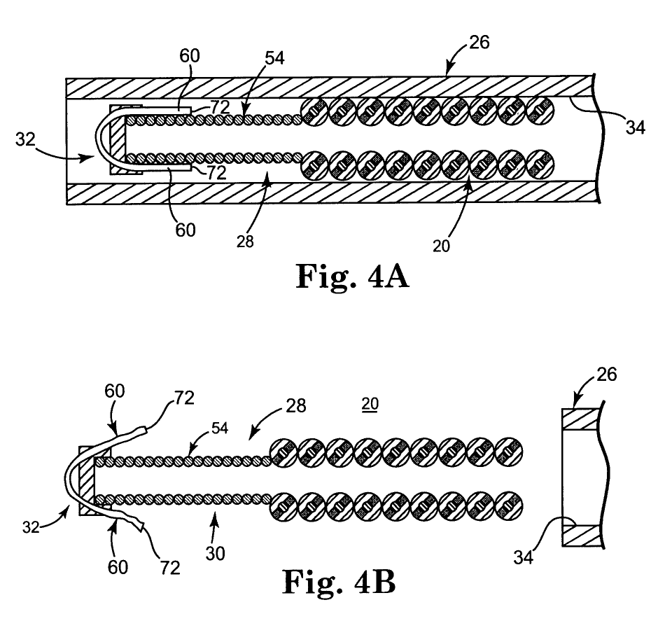

In general terms, the fixation assembly 32 includes at least one fixation member 60 and is coupled to the uncovered distal region 54 of the wire coil 40 so as to define (and be transitionable between) an expanded state (reflected, for example, in FIGS. 2 and 3) and a contracted state (see, e.g., FIG. 4A). With the one embodiment of FIGS. 2 and 3, the fixation assembly 32 includes a cap 62 maintaining a plurality of the fixation members 60 that are otherwise in the form of tines. More particularly, the tines 60 are formed by opposing legs of a suture or suture material 64, such that the tines 60 are highly pliable. Alternatively, other surgically safe, pliable polymeric materials can employed to form the tines 60. For example, the tines 60 can be formed of an absorbable material (e.g., a bio-resorbable suture material, an absorbable sponge material, etc.). Further, while two of the tines 60 are shown in FIGS. 2 and 3 has being formed by a single suture 64, in other embodiments, separate sutures (or other polymeric material strands) are provided to form a corresponding one of the tines 60. Along these same lines, while two tines 60 are shown, in alternative embodiments, any other number of the tine(s) 60, either greater or lesser, can be provided.

Regardless of the exact material and format of the tines 60, each tine 60 is generally defined by a base end 70 and a free end 72. The base end 70 is coupled to the uncovered distal region 54 of the wire coil 40, and thus at or distal the electrode 30, via the cap 62. With this construction, in the absence of an external force being placed upon the tine 60/free end 72, the free end 72 can move radially outwardly relative to the wire coil 40 to define an expanded state of the fixation assembly 32; conversely, the free end 72, and thus the tine 60 as a whole, can be forced against uncovered distal region 54 to define a contracted state as described in greater detail below. With this in mind, however, in one embodiment, the tine 60 has a thickness approximating a thickness of the non-conductive material 42 (e.g., a thickness of each of the tines 60 is not more than 0.005 inch greater than a thickness of the non-conductive material 42, more preferably has the same thickness or is thinner than the non-conductive material 42). When forced against the uncovered distal region 54, then, an overall diameter defined by a combination of the wire coil 40 and the tine(s) 60 approximates (e.g., plus or minus 0.005 inch) the diameter D2 defined by a combination of the wire coil 40 and the non-conductive material 42. With this one embodiment, then, the fixation assembly 32 is configured to facilitate passage through a conventional foramen needle lumen (e.g., the needle lumen 34 (FIG. 1)) by not overtly enlarging an effective outer diameter of the lead 20. Along these same line, in one embodiment each of the tines 60 has a length (i.e., distance or extension between the base and free ends 70, 72) that is less than a longitudinal length of the uncovered distal region 54 such that when forced on to the uncovered distal region 54, the tine(s) 60 do not extend to or overlap the non-conductive material 42 in a manner that might otherwise create a larger effective diameter for the lead 20 and rendering use thereof with the needle 26 more difficult.

The cap 62 is formed of a material suited for fixation to the wire coil 40, and in one embodiment is metal (e.g., stainless steel) that can be attached to the uncovered distal region 54 of the wire coil 40 via welding. Alternatively, the cap 62 can be formed from a variety of other materials and/or can be secured to the wire coil 40 using other manufacturing techniques (e.g., adhesive, over-molding, etc.). With respect to the one embodiment in which the cap 62 is metal, the direct coupling to the wire coil 40 can result in the cap 62 further serving as part of the electrode 30. Conversely, where the cap 62 is formed of an electrically non-conductive material, the cap 62 may slightly lessen an effective length of the electrode 30 by covering a short segment of the uncovered distal region 54. Regardless, the cap 62 is configured to capture the fixation member (e.g., tine(s)) 60 relative to the distal end 46/uncovered distal region 54 and in one embodiment forms passages 80a, 80b (referenced generally) through which the suture 64 (with the one embodiment of FIGS. 2 and 3) extends and is secured. Alternatively, the cap 62 can have a wide variety of other constructions capable of connecting or capturing the fixation member(s) 60 relative to the wire coil 40, and in particular relative to the uncovered distal region 54.

As alluded to above, the fixation assembly 32 is, in terms of one or both of configuration or assembly to the wire coil 40, capable of defining, and transitioning between, the contracted state and the expanded state. Relative to the one embodiment of FIGS. 2 and 3, the contracted and expanded states can be best understood with reference to FIGS. 4A and 4B. In particular, FIG. 4A illustrates the fixation assembly 32 in the contracted state. The fixation member(s) 60, including the respective free ends 72 thereof, are forced toward and/or lie against the uncovered distal region 54. For example, due to the highly pliable nature of the tines/fixation members 60 in accordance with one embodiment, the tines/fixation members 60 will readily assume the contracted state of FIG. 4A, especially in the presence of a constraining force being placed upon the tines 60. In the contracted state, the lead 20 is readily inserted within the small diameter lumen 34 of the needle 26 as shown in FIG. 4A. In this regard, and in one embodiment, the fixation assembly 32 is configured such that as the lead body 28 is co-axially inserted into the needle lumen 34 and advanced distally, the fixation members 60 contact the lumen 34 wall such that the needle 26 forces or constrains the fixation assembly 32 to transition to the contracted state of FIG. 4A.

Conversely, in the absence of an external force, the fixation assembly 32 is capable of self-transitioning from the contracted state and can assume the expanded state of FIG. 4B. For example, FIG. 4B illustrates the lead body 28 removed or deployed from the needle lumen 34. Once deployed, the needle 26 no longer exerts a constraining force on to the fixation members/tines 60. As such, the free end 72 of each of the tines 60 can move radially outwardly away from the wire coil 40 (e.g., when the lead body 28 has been implanted and is subjected to a proximal pulling force, the tines 60 will interact with bodily tissue and splay to the expanded state of FIG. 4B); alternatively or in addition, one or more of the tine(s) 60 can have a shape memory characteristic causing self-transitioning to or toward the expanded state. In the expanded state, the fixation assembly 32 focuses a resistance force to lead body 28 migration directly at the electrode 30/uncovered distal region 54, unlike conventional implantable stimulation electrode designs in which the anchor mechanism, if provided, is located well away from (proximal) the electrode(s).

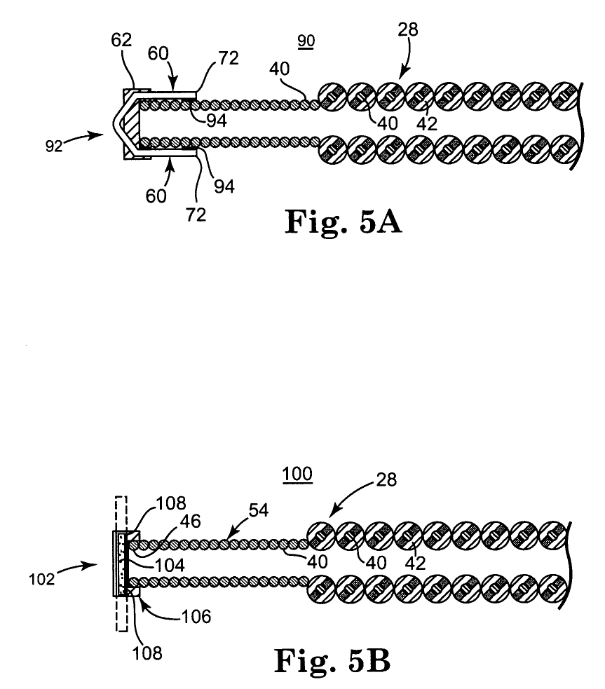

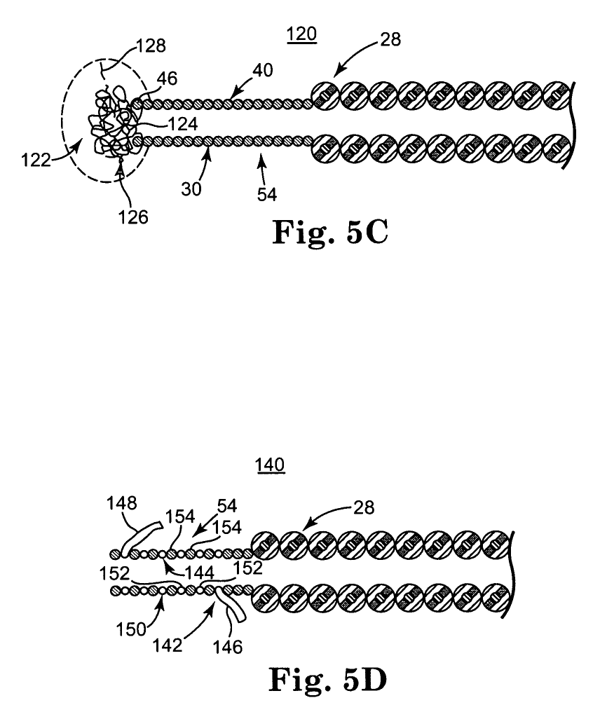

The lead 20, and in particular the fixation assembly 32, described above is but one acceptable configuration in accordance with principles of the present invention. For example, a portion of an alternative embodiment lead 90 is provided in FIG. 5A and includes the lead body 28 as previously described (including the wire 40 and the non-conductive material 42) and a fixation assembly 92. The fixation assembly 92 is highly similar to the fixation assembly 32 (FIGS. 2 and 3) previously described, and includes the fixation members 60 (in the form of tines) and the cap 62. In addition, the fixation assembly 92 further includes a bonding agent or adhesive 94 (a thickness of which is exaggerated in the view of FIG. 5A) temporarily adhering the tines 60, for example the free ends 72 thereof, to the contracted state reflected in FIG. 5A. In one embodiment, the bonding agent 94 is formulated to dissolve in the presence of liquids (e.g., bodily fluids). For example, the bonding agent 94 can be a sugar or mannitol-based adhesive, although other surgically safe formulations are equally acceptable. With this configuration, the bonding agent 94 maintains the tines 60 in the contracted state for ready insertion through the needle lumen 34 (FIG. 1). When the lead 90 is subsequently deployed from the needle lumen 34 at a surgical site within a patient, the bonding agent 94 will interact with bodily fluids and dissolve, thereby releasing the tines 60; the tines 60, in turn, are then able to transition to the expanded state as previously described. In related embodiments, the dissolvable bonding agent 94 can be employed with entirely different fixation assembly 92/fixation member 60 designs, for example with fixation member(s) 60 that are otherwise not highly pliable and/or have distinct shape memory attributes.

A portion of yet another alternative embodiment lead 100 in accordance with principles of the present invention is shown in FIG. 5B. Similar to previous embodiments, the lead 100 includes the lead body 28 as previously described (including the wire 40 and the non-conductive material 42) along with a fixation assembly 102. The fixation assembly 102 includes a fixation member 104 in the form of an osmotic material capable of expanding in size upon absorbing liquid (e.g., sponge, hydrogel, etc.), and a cap 106. The cap 106 is similar to the cap 62 (FIGS. 2 and 3) previously described, and connects or captures the sponge 104 relative to the distal end 46, and in particular the uncovered distal region 54, of the wire coil 40. With this configuration, the contracted state of the fixation assembly 102 is characterized by the osmotic material 104 being relatively dry or free of liquid such that the fixation member 104 has a relatively small overall size as shown in FIG. 5B. In the contracted state, then, the lead 100 is readily insertable through the small diameter needle lumen 34 (FIG. 1). Once deployed at a surgical site within a patient, the osmotic material 104 will begin to absorb water and/or other bodily fluids, thus expanding in size and self-transitioning to the expanded state (shown with dashed lines in FIG. 5B). The cap 106 is adapted to permit and/or direct sponge expansion to occur in a generally radially outward fashion relative to the wire coil 40, such as via passages 108 (shown generally in FIG. 5B).

A portion of yet another alternative embodiment lead 120 in accordance with principles of the present invention is shown in FIG. 5C and includes the lead body 28 as previously described and a fixation assembly 122. With this one embodiment, the fixation assembly 122 consists of one or more sutures 124 formed into a bundle 126 (referenced generally in FIG. 5C). The suture bundle 126 is connected (e.g. intertwined and/or secured with knots) to the distal end 46 of the wire coil 40. Further, the suture bundle 126 is compressible to a relatively small effective outer diameter, sufficient for placement within the needle lumen 34 (FIG. 1). In this so-created contracted state, then, the suture bundle 126 can be inserted within the needle lumen 34 and directed there through via a distal pushing force applied to the lead 120 (and thus to the distal end 46/suture bundle 126 interface). Once deployed from the needle lumen 34, the fixation assembly 122 transitions to an expanded state. For example, upon removal of the constraining force otherwise imparted by the needle 26, the suture bundle 126 will self-transition to a larger effective diameter or size (represented schematically by dashed lines in FIG. 5C). Alternatively or in addition, one or more suture lengths 128 (shown schematically in FIG. 5C) can free extend radially outwardly relative to the wire coil 40 to provide additional resistance to migration of the lead 120, with this resistive force again being focused at or distal the electrode 30/uncovered distal region 54.

A portion of yet another alternative embodiment lead 140 in accordance with principles of the present invention is shown in FIG. 5D. The lead 140 includes the lead body 28 as previously described, along with a fixation assembly 142. With the one embodiment of FIG. 5D, the fixation assembly 142 includes a suture or strand of surgically safe material 144 defining proximal and distal segments 146, 148 and an intermediate segment 150 (referenced generally). The intermediate segment 150 is co-axially wound with the wire coil 40 to secure the intermediate segment 150 to the uncovered distal region 54. For example, individual windings 152 of the intermediate segment 150 can be interposed between adjacent windings 154 of the wire coil 40. Regardless, the proximal and distal segments 146, 148 extend outwardly from the wire coil 40, and thus serve as pliable fixation members in the form of tines. In alternative embodiments, only one of the proximal segment 146 or the distal segment 148 extends from the wire coil 40; in yet other embodiments, a plurality of sutures 144 are wound within the wire coil 40. In any event, the proximal and/or distal segments 146, 148 can be forced against the uncovered distal segment 54 to define a contracted state of the fixation assembly 142 (such as when the lead 140 is inserted within the needle lumen 34 (FIG. 1)), and, in the absence of an external force, can extend radially outwardly relative to the wire coil 40 to define an expanded state as previously described.

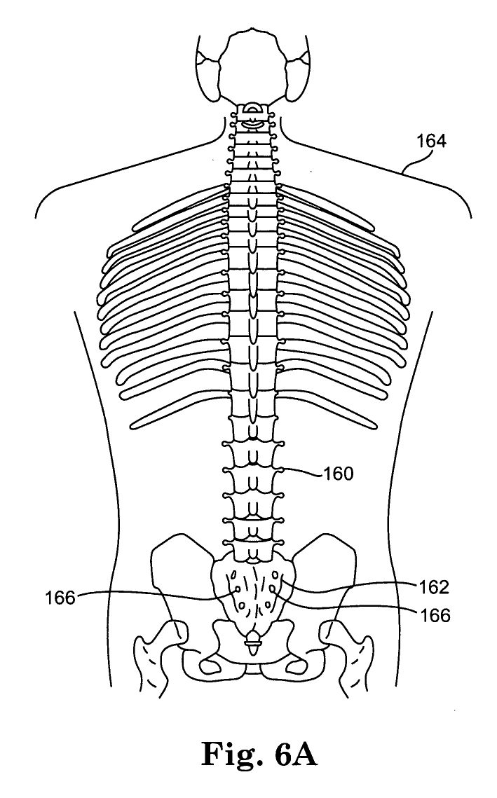

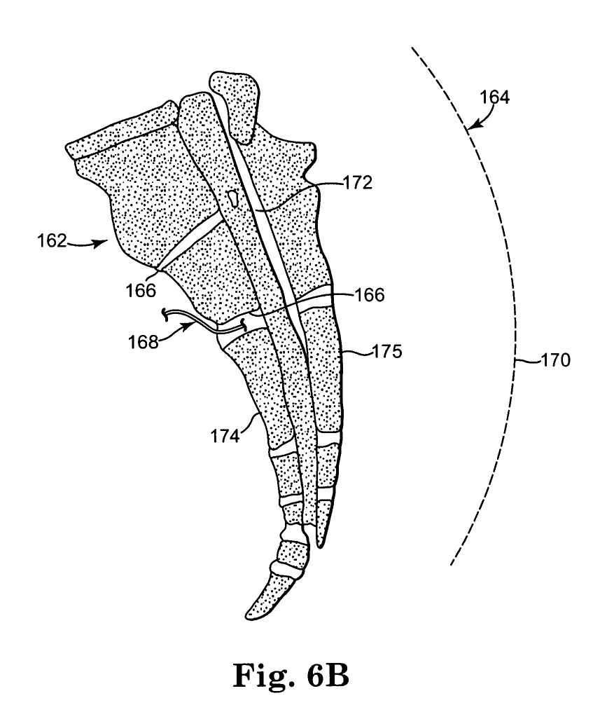

Returning to FIG. 1, the system 22 in accordance with principles of the present invention can be utilized to provide medical electrical stimulation from the external power source 24 to a wide variety of bodily structures via a percutaneous approach. For example, the system 22 can be deployed to stimulate one or more nerves of the nervous system. Alternatively, the system 22 can be used in other applications requiring electrical stimulation, such as procedures to rehabilitate muscle dysfunction by neuromodulation (e.g., functional electrical stimulation) of muscular behavior. In one embodiment, however, the system 22 is employed to provide electrical stimulation to a sacral nerve(s), for example as part of a peripheral sacral nerve simulation test or evaluation. In this regard, FIG. 6A is a posterior view of a spinal column 160 showing a location of a sacrum 162 relative to an outline of a patient's body 164. As shown, the sacrum 162 has a series of holes, known as foramina 166, there through. Each foramen 166 provides access to the sacral ventral nerves (not shown). This relationship is further illustrated in FIG. 6B whereby sacral nerves (a peripheral sacral nerve of which is illustrated schematically and generally referenced at 168) extend along the sacrum 162, generally opposite a dorsal surface 170 of the patient's body 164, and through or from a sacral canal 172. FIG. 6B further illustrates a pelvic surface 174 and a dorsal surface 175 of the sacrum 162.

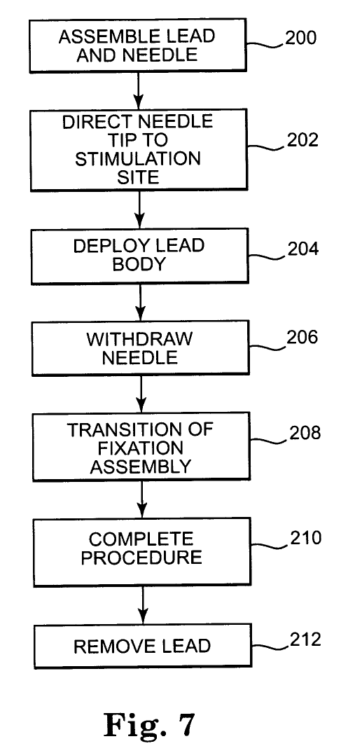

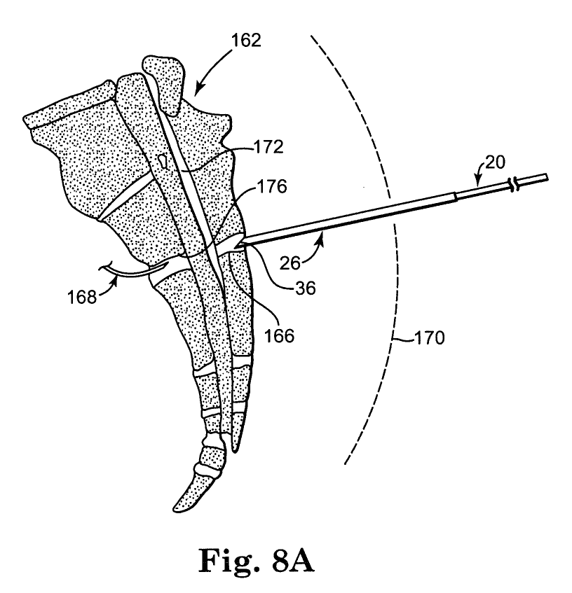

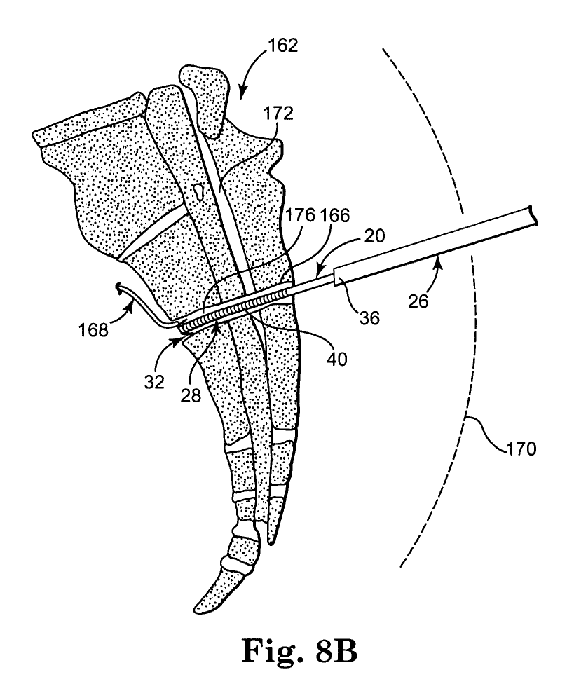

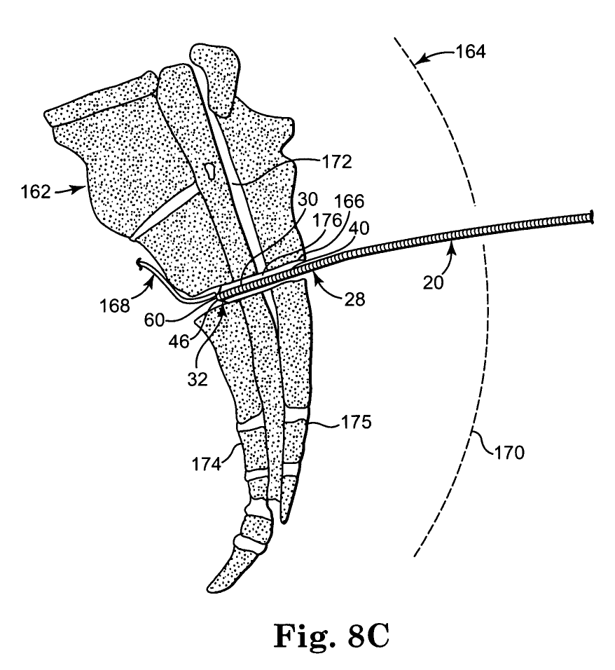

With the above anatomical description in mind, one method of using the lead 20 and associated system 22 to provide medical electrical stimulation to at least one of the sacral nerves 168 in accordance with principles of the present invention is provided by the flow diagram of FIG. 7, in conjunction with the views of FIGS. 4A and 8A-8C. As a point of reference, while the foregoing description relates to the system 22 incorporating the lead 20 configuration of FIGS. 2 and 3, the methodology is equally applicable using the alternative embodiment leads 90, 100, 120, and 140. Regardless, at step 200, the system 22 is assembled, including insertion of the lead body 28 into the needle lumen 34 as shown in FIG. 4A. Prior to and/or with insertion into the needle lumen 34, the fixation assembly 32 is positioned or transitioned to the contracted state. For example, with the one embodiment illustrated in FIG. 4A, the pliable tines 60 are, with insertion into the needle lumen 34, forced toward or against the uncovered distal region 54. Thus, the needle lumen 34 can have a small diameter (for example, the lumen diameter provided with a 20 gauge foramen needle available from Medtronic, Inc. of Minneapolis, Minn. under product numbers 041828 or 041829) appropriate for guiding a conventional PNE lead, and the fixation assembly 32 will not overtly impede passage of the lead body 28 there through. Though not shown, in some embodiments, a stylet can be employed to assist in directing the lead body 28 though the needle lumen 34.

At step 202, and with additional reference to FIG. 8A, the needle 26, and in particular a tip 36 thereof, is percutaneously directed toward the stimulation site or desired implantation site 176 (referenced generally in FIG. 8A). As a point of reference, the lead 20 can be loaded into the needle lumen 34 (FIG. 4A) following percutaneous delivery of the needle tip 36 (i.e., step 202 can occur prior to or in conjunction with step 200). Further, known techniques can be employed to identify the stimulation site 176. For example, the needle tip 36 can be an electrode (with a remainder of the needle 26 being electrically insulated with a non-conductive coating, such as parylene) that is periodically or continuously energized to locate one or both of the foramen 166 or the peripheral sacral nerve 168 to be electrically stimulated. To this end, and in one embodiment, the needle tip 36 can be sharpened (although in other embodiments, the needle tip 36 is not sharp) and the needle tip 36/needle 26 is sized and adapted to access the desired foramen 166. Regardless, the stimulation site 176 is characterized as being in sufficiently close proximity to the sacral nerve 168 in question such that electrical energy applied at the stimulation site 176 stimulates the sacral nerve 168 (as evidenced, for example, by the stimulation energy causing a known physical response in the patient such as involuntary toe or foot movement).

Upon identifying the stimulation site 176, at step 204 the lead body 28 is deployed from the needle tip 36 as generally shown in FIG. 8B. This can be accomplished in a variety of fashions, for example by proximally retracting the needle 26, distally advancing the lead 20 (via a stylet, for example), or both. At step 206, with the lead body 28 implanted at the stimulation site 176, the needle 26 is proximally withdrawn from the lead 20. Following deployment from the needle tip 26, the fixation assembly 32 transitions or is transitionable to the expanded state at step 208. For example, and with reference to the one embodiment of FIG. 8C, once released from the needle 26 (FIG. 8B), the tines 60 (one of which is illustrated in FIG. 8C) are no longer constrained against the wire coil 40, such that the corresponding free end 72 can move radially away from the wire coil 40. In some embodiments, the fixation assembly 32 is configured such that the free end 72 of one or more of the tines 60 will self-transition at least a slight distance away from the wire coil 40. In addition and/or alternatively, in the event a pulling force is applied proximally to the lead body 28, the free ends 72 will further splay outwardly away from the wire coil 40 due to an interface with bodily materials at the stimulation site 176. Regardless, the fixation assembly 32, in the expanded state, inhibits migration of the lead body 28, for example axial retrograde dislodgment of the lead body 28 back through the foramen 166, especially in a region of the electrode 30 due in part to the close proximity of the fixation assembly 32 to the electrode 30/distal end 46 of the lead body 28. As shown in FIG. 8C, at the stimulation or implantation site 176, the fixation assembly 32 has passed anteriorly beyond the dorsal surface 175 of the sacrum 162. In connection with the position and/or in being delivered to this position, the fixation assembly 32 can contact the peripheral sacral nerve 168 without damaging the nerve 168 due to the pliable nature of the fixation members 60 (in accordance with some embodiments). Further, the fixation assembly 32 is within, and in some embodiments anteriorly beyond, the foramen 166 such that the fixation assembly 32 interacts with the boney structure of the sacrum 162. In some embodiments, the fixation member(s) 60 contact or engage the pelvic surface 174. In other embodiments, the lead body 28 is positioned at the stimulation site 176 such that the fixation assembly 32 is located within the foramen 166 anteriorly proximate the dorsal surface 175.

Following implantation of the lead body 28 and removal of the needle 26, other activities are performed at step 210 to complete the procedure. For example, and with additional reference to FIG. 1, the proximal portion 48 of the wire 40 remains external the patient, and the proximal end 50 is electrically coupled to the external power source 24. In one embodiment, the power source 24 is a pulse generator, such as a Model 3625 InterStim® Test Stimulator available from Medtronic, Inc., although a number of other devices can be used as the external power source 24. Similarly, additional cable(s) (not shown) can be provided to effectuate electrical coupling of the proximal end 50 of the wire 40 to the power source 24 external the patient, along with a return electrode or ground pad (not shown) being applied to the patient's skin in accordance with some embodiments. Where the method of FIG. 7 is performed in conjunction with a sacral peripheral nerve evaluation stimulation test, the power source 24 operates over the course of, for example, 3-7 days, periodically applying a stimulation energy to the electrode 30 that in turn stimulates the sacral nerve 168 (FIG. 8C). The patient can make a record of various results, if any, of the stimulation for subsequent evaluation of whether a permanently-implanted nerve stimulation system is a viable option. Regardless, at step 212, the lead body 28 is removed or explanted from the stimulation site 176 and the patient by, for example, applying a gentle pulling force on to the proximal portion 48 of the wire 40, otherwise external the patient.

The medical implantable electrical lead, system and method of the present invention provides a marked improvement over previous designs. In particular, the fixation assemblies described herein provide direct support to the electrode/distal region of the lead body in resisting migration following implant, but are capable of assuming a highly compact contracted state. As such, when used in conjunction with a small diameter lead body (for example a PNE lead), the present invention facilitates temporary implantation through a small diameter needle.

Although the present invention has been described with reference to preferred embodiments, workers skilled in the art will recognize that changes can be made in form and detail without departing from the spirit and scope of the present invention. For example, while the implantable electrical lead has been described as including or providing a single electrode (and thus operable in a unipolar fashion), in other embodiments, the present invention is equally useful with a lead having a plurality of electrodes (e.g., a lead configured to provide bipolar operation).

What is claimed is:

1. An implantable medical electrical lead for applying electrical stimulation to bodily tissue of a patient from a power source located external the patient, the lead adapted to be introduced through, and released from, a needle having a lumen defining a diameter of not greater than 0.05 inch, the lead comprising:
a lead body including:
a wire defining a distal portion terminating at a distal end and a proximal portion terminating at a proximal end, the wire forming a wound coil along at least the distal portion, and the proximal end adapted to be electrically coupled to a power source,
an electrically non-conductive material covering a section of the distal portion, wherein the non-conductive material terminates proximal the distal end to a define an uncovered distal region of the wire coil,
wherein at least a segment of the uncovered distal region serves as a lead electrode; and
a fixation assembly coupled to the uncovered distal region, the fixation assembly including at least one fixation member;
wherein the fixation assembly is configured and assembled to the wire coil to be transitionable between a first, contracted state and a second, expanded state, a radially outward extension of the fixation member relative to the wire coil being greater in the expanded state than in the contracted state to inhibit axial dislodgement of the lead body.

2. The lead of claim 1, wherein in the contracted state, the fixation assembly and the wire coil combine to define an outer diameter of not more than 0.04 inch.

3. The lead of claim 1, wherein the fixation assembly is coupled to the distal end of the wire coil.

4. The lead of claim 1, wherein the fixation member is a pliable tine.

5. The lead of claim 4, wherein the fixation assembly includes a plurality of tines.

6. The lead of claim 4, wherein the tine is a surgical suture.

7. The lead of claim 4, wherein the tine is formed of a pliable polymeric material.

8. The lead of claim 4, wherein the tine has a thickness approximating a thickness of the non-conductive material.

9. The lead of claim 4, wherein the tine extends from the wire coil to a length that is less than a longitudinal length of the uncovered distal region of the wire coil.

10. The lead of claim 4, wherein the fixation assembly is configured such that in the contracted state, the tine lies against the uncovered distal region of the wire coil and in the expanded state, a free end of the tine is radially moveable away from the wire coil.

11. The lead of claim 10, wherein the fixation assembly further includes an adhesive temporarily bonding the free end to the wire coil in the contracted state and formulated to dissolve in the presence of liquids, the fixation assembly being transitionable to the expanded state upon dissolving of the adhesive.

12. The lead of claim 4, wherein the fixation assembly further includes:
a cap configured to be mounted over the distal end of the wire coil and further to maintain the tine relative to the wire coil upon assembly of the cap to the wire coil.

13. The lead of claim 12, wherein the tine is formed by a first segment of a suture extending through a first passage in the cap.

14. The lead of claim 13, wherein the suture includes a second segment extending through a second passage in the cap to provide a second tine.

15. The lead of claim 1, wherein the fixation member is an osmotic expanding material.

16. The lead of claim 15, wherein the sponge material is relatively free of water in the contracted state and absorbs water in transitioning to the expanded state.

17. The lead of claim 1, wherein the fixation assembly includes at least one suture formed as a compressed bundle in the contracted state.

18. The lead of claim 1, wherein the fixation assembly is a suture having proximal, intermediate, and distal segments, and further wherein the intermediate segment is wound within windings defined by the wire coil at the uncovered distal region.

19. A system for providing medical electrical stimulation to bodily tissue of a patient from a power source located external the patient, the system comprising:
a hollow needle defining a lumen having a diameter of not more than 0.05 inch; and
an implantable medical electrical lead slidably disposed within the needle lumen, the lead including:
a lead body including:
a wire defining a distal portion terminating at a distal end and a proximal portion terminating at a proximal end, the wire forming a wound coil along at least the distal portion, and the proximal end adapted to be electronically coupled to a power source,
an electrically non-conductive material covering a section of the distal portion, wherein the non-conductive material terminates proximal the distal end to define an uncovered distal region of the wire,
wherein at least a segment of the uncovered distal region serves as a lead electrode, and
a fixation assembly coupled to the uncovered distal region, the fixation assembly including at least one fixation member,
wherein the fixation assembly is configured and assembled to the wire coil to be transitionable between a first, contracted state and a second, expanded state, a radially outward extension of the fixation member relative to the wire coil being greater in the expanded state that in the contracted state to inhibit axial dislodgment of the lead body.

20. The system of claim 19, wherein the fixation assembly is configured to be forced to the contracted state when the lead body is inserted into the needle lumen.

21. The system of claim 19, wherein the needle lumen has a diameter of not more than 0.04 inch.

22. The system of claim 19, wherein the needle is a 20 gauge needle.

23. The system of claim 19, wherein the fixation assembly is configured to be self-transitionable from the contracted state to the expanded state upon release of the lead body from the needle lumen.

24. The system of claim 19, wherein the system is configured for performing a peripheral sacral nerve stimulation procedure such that the lead body is a peripheral nerve evaluation lead and the needle is adapted to percutaneously access a sacral foramen.

25. A method of providing electrical stimulation to bodily tissue of a patient at a stimulation site via a power source external the patient, the method comprising:
providing an implantable medical electrical lead including:
a lead body comprising:
a wire defining a distal portion terminating at a distal end and a proximal portion terminating at a proximal end, the wire forming a wound coil along at least the distal portion,
an electrically non-conductive material covering a section of the distal portion, wherein the non-conductive portion terminates proximal the distal end to define an uncovered distal region of the wire,
wherein at least a segment of the uncovered distal region serves as a lead electrode,
a fixation assembly coupled to the uncovered distal region and including at least one fixation member,
wherein the fixation assembly is configured and coupled to the wire coil to define a contracted state and an expanded state, a radially outward extension of the fixation member relative to the wire coil being greater in the expanded state than in the contracted state;
slidably disposing the lead body within a lumen of a needle, the lumen having a diameter of not greater than 0.05 inch;
wherein the fixation assembly is in the contracted state when the lead body is within the lumen;
percutaneously directing a distal tip of the needle toward the stimulation site;
deploying the lead body from the distal tip to implant the lead body at the stimulation site;
transitioning the fixation assembly from the contracted state to the expanded state;
proximally withdrawing the needle from the lead such that the proximal portion of the wire is external the patient; and
electrically coupling the proximal end of the wire to a power source external the patient;
wherein following implantation, the fixation assembly in the expanded state inhibits axial retrograde migration of the lead body from the stimulation site.

26. The method of claim 25, wherein in the expanded state, the fixation assembly applies a dislodgement resistance force directly on to the uncovered distal region of the wire coil.

27. The method of claim 25, wherein the fixation assembly self-transitions from the contracted state to the expanded state upon deployment of the lead body from the needle.

28. The method of claim 27, wherein self-transitioning of the fixation assembly is characterized by a free end of the fixation member being released relative to the wire coil.

29. The method of claim 27, wherein self-transitioning of the fixation assembly is characterized by the fixation member absorbing bodily fluids.

30. The method of claim 25, wherein the method is performed as part of a peripheral sacral nerve stimulation procedure such that the stimulation site is in operative proximity of a sacral nerve, and further wherein percutaneously directing a distal tip of the needle includes:
directing the needle tip into a sacral foramen.

31. The method of claim 25, further comprising:
applying a pulling force on to the proximal portion of the wire external the patient to explant the lead body from the stimulation site.
